SEQUENCE LISTING (2 sequences)

SEQ ID NO: 1 - DNA, 6879 bp, Mus musculus
atgttatacaaccagccagaccagaaatatgatgaagagaatcttccaataccaaattctctacgttgtgagctcttacttgttttgaaaactcagtggccctttgatccagaattttgggattggaaaactttgaagcgccagtgtcttgctctcatgggagaagaggcgtctattgtgtcctcaattgatgaactgaatgacagtgaagtctatgagaaagtagactaccagggtgaaaggggagacacatctgtgaatggcctttctgctgctggacttggtactgattctggcctgctgatggatactggtgatgaaaagcagaagaagaaagagataaaagaattaaaagatagggggtttatatctgctaggtttaggaattggcaagcctacatgcagtattgtttgctctgtgacaaagaattccttggacacagaatagtacggcatgctcaaaaacattacaaagatgggatttacagctgtcccatatgtgcaaagaattttaattctaaagactcgtttgtccctcatgttaccctgcatgttaaacagtctagtaaagagagactagcagctatgaagccattaagaagattgggaaggcctcctaaaatcacagccacccatgaaaatcaaaagactaatattaatactgtggctaaacaggaacagcgacccataaaaaagaatagtctttattcaacagatttcatagtgtttaatgacaacgatggttcagatgatgaaaatgacgacaaggacaagtcttatgagcccgaggtgatccctgtccagaaaccagtacctgttaatgagtttaattgtcctgtgaccttttgtaaaaagggctttaagtacttcaaaaatttaattgctcatgtgaaaggccataaggatagtgaagatgccaaacgctttcttgaaatgcaaagcaagaaagtcatttgccagtactgtagaaggcactttgtaagcgtcactcatctcaatgatcacttacaaatgcactgtggcagtaagccatatatatgtatacagatgaaatgtaaggctggttttaatagttacgcagagctgttagcccaccgaaaggagcatcaagtctttagagcaaagtgcttatttccaaaatgtggcagaattttttcccaagcttatttactgtatgatcatgaggcccaacattataatacgtacacgtgcaagttcacaggttgtggtaaggtgtaccgttctcagagcgagatggagaagcaccaggatggccacagtcatcctgaaacagggctgcctcctgaagaccagcttcagccatctggaaatgatgtgaatccggactcaggagcgacggctgcaggaggaaggtccgagaacagcattgacaagaacctgggttcaaacagaagtgcagattgggagaaaaacagagcagagccagctgtgactaaacacggccagatctctgccgctgaactcaggcaagctaacataccattgtcaaatggtctggaaacccgtgataatactactgttcttcggaccaatgaagtagctgtgtccatcaaggtgtctgtcaaccatggggtagagggtgactttggaaagcaagaaaacctaaccatggaaggcactggtgagccgctgatcacagatgtgcataaaccaggtataggtgctggggtccagttatgtcatccaggtttccaagaaaagaaaggtcacgagtgcctgaacgaagcccagaattctttatcaaactcagaatcactgaagatggatgaccttaacccacaaagcttagaaagacaggtgaacactctgatgaccttttctgtacaaaatgaggcaggacttgaagacaattcacaaatttgcaagtttgaatgtggaggtgatgttaaaacctcatccagcctttatgatttacctcttaagacactagaaagtatcacatttgttcagtcacagcccgacctaagcagtccgttgggatctccatcagtacctccaaaagctccaggtcagaagttcagctgccaggttgagggatgcactcgaacatataactcttcacagagtattggaaaacacatgaagacagcacaccctgaccaatatgctgcttttaaactgcagcgcaagacgaaaaaaggtcagaaatctaacaacttaaatacaccaaatcatggaaagtgtgtttattttttgccatcacaagtgagcagctctaatcatgctttttttacaccacagaccaaagccaatgggaaccctgcctgttcagcccaggtgcagcatgtctcgccttccattttcccagctcatttagcaagtgtatcagctccattgttaccctcagtggaaagtgtcctaagtccaaatataccttctcaggataaacatggacaagatggcatattatgttcacaaatggaaaatttgtcttatgctcccttgccagcacaaatggaagatctaaccaagacagttttgcctttgaatattgacagcggctcagatccgtttcttcctttacccacagaaaatagctctctcttctcttcaccagcagacagtgagaataattctgttttttcccaactggaaaatagtacaaatcattatccctcccagacggatggaaacataaattcctcttttctgaaaggaggcagcagtgaaaatggagtttttccttcccaagtaagttctgcagatgacttcagtagcaccagtgcccaaccgtctacacctaagaaagtgaaaaaagaccgtggtcgaggcccaaatgggaaggaaagaaaacccaagcacaacaaaagggctaaatggcctgcgattatcagggatgggaaattcatctgtagcaggtgttacagggctttcaccaaccccaggtccctgggtggacacctgtctaaaaggtcttactgcaaaccactggatggagcagaaatagcacaggaacttctacagaccaacagacagccttccctcctagctagcatgattctctccacaagtgcagtaaatatgcaacagccgcaacagtctaactttaatccagaaacatgctttaaagacccatcattcctgcaacttcttaatgtggaaaatcgtccaacctttttaccaagtacatttccaagatgtgacgtgagtaactttaatgccagtgttagtcaggaaggcagtgaaattattaagcaggctttagaaactgctggcattcccagcacgtttgagagtgccgaaatgctttctcaggttgttccaataggcagtgtctccgatgcagcacaagtcagtgcagcggggatgccagggccacctgtgacacccttgttacagactgtttgccacccaaacacctcaccatcaaaccagaatcaaacgccaaattccaaaaccctcaaagaatgtaacagtttgcctctctttacaacaaatgatttactgctaaagactattgaaaatggcttgtgctccaattcattcagtagttctactgaaccaccacaaaattttaccaataatagtgcacatgtttctgttataagtgggcctcagaatacaagatccagtcatttgaataaaaaaggaaatagtgcatctaagaagagaaaaaaagttgctcctgcagtaagtgtatctaatacttcccaaaatgtgctaccaactgatttaccagtgggccttccatcgaagaatcttacagtccctgataccaacacacggtcagacatgaccccagattgtgaacctcgggctttggtggaaaatctcacacagaaattaaataacattgacaatcatttgtttataactgatgtaaaagagaactgtaaagccagtcttgagccccatacaatgttaacccctttaacattaaaaacggaaaacggcgattcccgaatgatgcctttgagttcatgcacaccagtgaattctgatttgcagatttctgaagataatgttattcagaactttgagaagactcttgaaattattaaaactgctatgaattctcaaatacttgaggtaaaaagtggatctcagggtactggtgagacaacacagaatgctcagataaattacagcatgcaacttccctcagtaaactctatcccagatagcaagctgcctgatgcttctcagtgctcctctttcctaactgtaatgccaacaaagtctgaagcattacataaggaggatcaaatacaggacattttagagggtttgcaaaacttaaaactagaaaatgacacttctgctccagcttcccagagtatgctaatgaacaaatcagtagcactgtcccctactcctactaaatcaactccaaatattgtagtccagccagtacccgaagtgatacatgttcagcttaatgacagagttaataagccctttgtgtgtcaaaaccaaggctgtaactacagtgctatgacaaaggatgccctgtttaaacactatggtaaaatccatcagtatactccagagatgattcttgaaattaagaagaatcaattaaaatttgctccatttaaatgtgtagtaccttcatgtaccaaaacatttacaagaaattctaatctccgggcacactgtcagttggtgcatcattttacaatagaagaaatggtaaagctaaaaataaaaaggccctatggaagaaaatctcagagtgaaaatttgtcatctccacagaataatcaagtgaagaaacagccatccatggccgaggaaacaaaaactgagtcacaaccagccttcaaggtaccagcagcaacaggtgatgctgcacttgctaatgcaacagtaatcccagaaaaacaacttgcagaaaaaaaaagtcctgagaaaccagaaagttcttcacagcctgtcacatcttctgctgaacaatataatgcaaatcttgcaaacctaaaaaccaaaggaaggaaaaataagaggcatagaaaagaaaaggaagaaaaacgggaaaagaatccagtttcccaggcctttgaacttccaacaaaatacagttcgtacagaccttactgctgtgtccaccagggatgctttgctgcttttacaatacagcaaaacttgattcttcattaccaggctgtacataaatcaaatcttcctacattttctgcagaggttcaagaggaaagtgaagctgttaaagaaagtgaagaaactgaaccgaaacaatcaatgaaagaatttaggtgtcaggtgagtgactgttctaggattttccaagcaattactggcctaatacagcactacatgaaacttcatgaaatgacccccgaggaaattgaaagcatgactgctgctgtggatgttggcaaatttccatgtgatcagttggagtgtaagttgtcttttacaacatacctgagctatgttgttcatcttgaggtagaccatggaattggaacaaggacaagtaaggcagaagaagatggcatatacaagtgtgactgtgagggctgtgacaggatatatgccactcggtctaatcttctccgacacatctttaataaacataatgacaagcataaagcccatctgattcggccaagaaaattaactggccaggaaaatatatcaagtaaggcaaaccaagaaaaatcaaagtctaaacatcggacaacaaaacccaacagatccgggaaagacggaatgaaaatgccaaagacaaagcgaaagaaaaaaagtaatttagaaaacaagagcgcaaaagtagtgcagattgaggaaaataagccttattctctaaagcgtgggaagcacgtgtattccataaaggctaggaatgatgccttggcagagtgtacaagcaaatttgtgacacagtatccatgtatgataaaagggtgtacttcagtcgttacaagtgaaagcaatatcatcagacattataagtgtcataagttgtccagggcatttacatcacaacaccgcaacattcttattgtctttaagcgatatggcaacccacaaggaagggaaatctctgagcaagaagatgaaaagaatgataagaaaggtcctgattcatctgttttagagaaaaatgataactcggaaccagctgctgctccacaggaagaaggtagaaaaggtgaaaaggatgagatggatgagttaacagaattatttattacaaagttaataaatgaagacagcacaaatgcagaaaaccaaggcaataccactttaaagggaaataacgaatttcaggagcatgattcctgcacatcagaaagacaaaagcctggtaatttgaagagagtttataaagaaaaaaacactgtgcagagtaagaaacggaagattgataaaactgagccagaagtatccttggtggtaaataatacacggaaagaggaagagcctgccgtagcagttcagaccactgaggagcatcctgcatcctttgactggagctccttcaagcctatgggatttgaagcatcctttctgaagtttcttgaagagtctgcagtgaagcagaagaaaaatagtgacagagaccattcaaacagtggaagtaaaagaggatcccattccagctccagaagacatgttgataaggctgctgtggctggtagcagtcatgtgtgttcctgtaaagacagtgaaatctttgtacagtttgccaacccctcaaagcttcagtgcagtgagaatgtaaaaattgttttagacaagactcttaaagatcgctctgagcttgtcctaaaacagcttcaggaaatgaaacctactgtcagtctaaaaaaacttgaagtactatccaatagtccagataggactgttttaaaagaaatcagtataggtaaagccacgggcagagggcagtac

SEQ ID NO: 2 - protein, 2293 aa, Mus musculus
MLYNQPDQKYDEENLPIPNSLRCELLLVLKTQWPFDPEFWDWKTLKRQCLALMGEEASIVSSIDELNDSEVYEKVDYQGERGDTSVNGLSAAGLGTDSGLLMDTGDEKQKKKEIKELKDRGFISARFRNWQAYMQYCLLCDKEFLGHRIVRHAQKHYKDGIYSCPICAKNFNSKDSFVPHVTLHVKQSSKERLAAMKPLRRLGRPPKITATHENQKTNINTVAKQEQRPIKKNSLYSTDFIVFNDNDGSDDENDDKDKSYEPEVIPVQKPVPVNEFNCPVTFCKKGFKYFKNLIAHVKGHKDSEDAKRFLEMQSKKVICQYCRRHFVSVTHLNDHLQMHCGSKPYICIQMKCKAGFNSYAELLAHRKEHQVFRAKCLFPKCGRIFSQAYLLYDHEAQHYNTYTCKFTGCGKVYRSQSEMEKHQDGHSHPETGLPPEDQLQPSGNDVNPDSGATAAGGRSENSIDKNLGSNRSADWEKNRAEPAVTKHGQISAAELRQANIPLSNGLETRDNTTVLRTNEVAVSIKVSVNHGVEGDFGKQENLTMEGTGEPLITDVHKPGIGAGVQLCHPGFQEKKGHECLNEAQNSLSNSESLKMDDLNPQSLERQVNTLMTFSVQNEAGLEDNSQICKFECGGDVKTSSSLYDLPLKTLESITFVQSQPDLSSPLGSPSVPPKAPGQKFSCQVEGCTRTYNSSQSIGKHMKTAHPDQYAAFKLQRKTKKGQKSNNLNTPNHGKCVYFLPSQVSSSNHAFFTPQTKANGNPACSAQVQHVSPSIFPAHLASVSAPLLPSVESVLSPNIPSQDKHGQDGILCSQMENLSYAPLPAQMEDLTKTVLPLNIDSGSDPFLPLPTENSSLFSSPADSENNSVFSQLENSTNHYPSQTDGNINSSFLKGGSSENGVFPSQVSSADDFSSTSAQPSTPKKVKKDRGRGPNGKERKPKHNKRAKWPAIIRDGKFICSRCYRAFTNPRSLGGHLSKRSYCKPLDGAEIAQELLQTNRQPSLLASMILSTSAVNMQQPQQSNFNPETCFKDPSFLQLLNVENRPTFLPSTFPRCDVSNFNASVSQEGSEIIKQALETAGIPSTFESAEMLSQVVPIGSVSDAAQVSAAGMPGPPVTPLLQTVCHPNTSPSNQNQTPNSKTLKECNSLPLFTTNDLLLKTIENGLCSNSFSSSTEPPQNFTNNSAHVSVISGPQNTRSSHLNKKGNSASKKRKKVAPAVSVSNTSQNVLPTDLPVGLPSKNLTVPDTNTRSDMTPDCEPRALVENLTQKLNNIDNHLFITDVKENCKASLEPHTMLTPLTLKTENGDSRMMPLSSCTPVNSDLQISEDNVIQNFEKTLEIIKTAMNSQILEVKSGSQGTGETTQNAQINYSMQLPSVNSIPDSKLPDASQCSSFLTVMPTKSEALHKEDQIQDILEGLQNLKLENDTSAPASQSMLMNKSVALSPTPTKSTPNIVVQPVPEVIHVQLNDRVNKPFVCQNQGCNYSAMTKDALFKHYGKIHQYTPEMILEIKKNQLKFAPFKCVVPSCTKTFTRNSNLRAHCQLVHHFTIEEMVKLKIKRPYGRKSQSENLSSPQNNQVKKQPSMAEETKTESQPAFKVPAATGDAALANATVIPEKQLAEKKSPEKPESSSQPVTSSAEQYNANLANLKTKGRKNKRHRKEKEEKREKNPVSQAFELPTKYSSYRPYCCVHQGCFAAFTIQQNLILHYQAVHKSNLPTFSAEVQEESEAVKESEETEPKQSMKEFRCQVSDCSRIFQAITGLIQHYMKLHEMTPEEIESMTAAVDVGKFPCDQLECKLSFTTYLSYVVHLEVDHGIGTRTSKAEEDGIYKCDCEGCDRIYATRSNLLRHIFNKHNDKHKAHLIRPRKLTGQENISSKANQEKSKSKHRTTKPNRSGKDGMKMPKTKRKKKSNLENKSAKVVQIEENKPYSLKRGKHVYSIKARNDALAECTSKFVTQYPCMIKGCTSVVTSESNIIRHYKCHKLSRAFTSQHRNILIVFKRYGNPQGREISEQEDEKNDKKGPDSSVLEKNDNSEPAAAPQEEGRKGEKDEMDELTELFITKLINEDSTNAENQGNTTLKGNNEFQEHDSCTSERQKPGNLKRVYKEKNTVQSKKRKIDKTEPEVSLVVNNTRKEEEPAVAVQTTEEHPASFDWSSFKPMGFEASFLKFLEESAVKQKKNSDRDHSNSGSKRGSHSSSRRHVDKAAVAGSSHVCSCKDSEIFVQFANPSKLQCSENVKIVLDKTLKDRSELVLKQLQEMKPTVSLKKLEVLSNSPDRTVLKEISIGKATGRGQY

(19) United States
(12) Patent Application Publication
(10) Pub. No.: US 2004/0186068 A1
(43) Pub. Date: Sep. 23, 2004

(54) Growth hormone transcription factor

(76) Inventor: David L. Hurley, New Orleans, LA (US)

Correspondence Address: DAVID L. HURLEY, 1128 FERN STREET, NEW ORLEANS, LA, 70118, US

(21) Appl. No.: 10/032,438

(22) Filed: Dec. 31, 2001

(51) Int. Cl. A61K 48/00; C07H 21/04; C12N 9/22

(52) U.S. Cl. 514/44; 435/325; 536/23.2; 435/69.1; 435/320.1; 435/199

(57) ABSTRACT

Taught is how to make a growth hormone transcription factor that is useful for the control of gene expression and growth rate in organisms.

### CROSS-REFERENCE TO RELATED APPLICATION

The invention described in the current application was also disclosed in provisional application 60/258,237, filed Dec. 25, 2000, from which priority is claimed.

### STATEMENT REGARDING FEDERALLY SPONSORED RESEARCH

Research support which led to the making of the present invention was provided in part by funding from the National Science Foundation under Grant No. IBN-9600805. Accordingly, the federal government may possess certain statutory rights to the invention.

### FIELD OF THE INVENTION

The invention relates to a growth hormone transcription factor.

### DESCRIPTION OF RELATED ART

Cell-specific gene expression leads to the exclusive production of secreted hormones by selected cell types in the anterior pituitary. These hormones are produced in differentiated cells through selective processes of transcriptional and translational control. In addition, each cell type displays a distinctive pattern of differentiation during development, and specific neuroendocrine regulation.

Transcriptional control of the growth hormone (GH) gene resides primarily in the promoter region that contains multiple transcription factor response elements. Pit-1, also known as GHF-1, is a prototypical POU-domain protein which was isolated by virtue of its ability to activate transcription of the GH gene by binding to the GH promoter at two sites. Pit-1 was also found to regulate transcription of the prolactin and TSH beta-subunit genes. Expression of all these genes is pituitary-specific, implying a central role of Pit-1 as a transcription factor controlling pituitary development and differentiation. The important role of Pit-1 in the regulation of anterior pituitary differentiation was confirmed by the identification of Pit-1 mutations as the causes of congenital hypopituitary dwarfism in the Snell dwarf mouse and in humans.

Because Pit-1 promotes transcription of three distinct hormones, other transcription factors must establish the specificity of GH production in somatotrophs. One such protein was isolated by virtue of its binding to an 18 base pair (bp) Z-box response element (ZRE) conserved among mammals in the GH promoter. The protein that bound to the ZRE contained 15 consensus sequences for DNA-binding zinc fingers, and was named Zn-15. It was also shown that in synergy with Pit-1, Zn-15 activates GH transcription 100-fold above basal levels. The importance of Zn-15 in the physiological regulation of GH gene expression was shown when mutations in the ZRE in the GH promoter abrogated pituitary expression of a reporter gene in transgenic animals. See Lipkin et al., Genes Devel. 7, 1674.

Although Zn-15 plays an important synergistic role in GH transcriptional control, only the C-terminal portion appears to be necessary for this synergism. Zinc fingers IX, X and XI have been shown to bind to the ZRE in the GH promoter, but DNA binding elements recognized by the remaining 12 zinc fingers of rat Zn-15 have not been found. Some of these fingers in the rat are separated by long linker regions of 20 or more amino acids, a structural feature evidently determinative of DNA binding or transcriptional activation. Zn-15 protein also can bind to a subset of thyroid response elements using fingers IX-XI, and may bind to RNA as well as DNA using the zinc fingers in the N-terminus of the protein. In order to effect cell type-specific regulation of GH, appropriate use must be made of the linkers and zinc fingers which do not bind the ZRE. See Tuggle and Trenkle, Domes. Anim. Endocrinol. 13, 1.

### BRIEF SUMMARY OF THE INVENTION

The invention relates to a growth hormone transcription factor that is useful for the control of gene expression and growth rate in organisms.

### DETAILED DESCRIPTION OF THE INVENTION

The present invention is useful, inter alia, for the control of cell-type specific regulation of GH in mammals, including, importantly, the mouse. Pituitary differentiation has been extensively studied in the mouse, where both transgenic and spontaneous GH-deficient mutants are available. To make a representative embodiment of the present invention, a mouse cDNA, hereinafter called mouse Zn-16 or mZn-16, was isolated. Expression of mouse Zn-16 mRNA was detected in 1 day old normal and GH-deficient Ames dwarf (Prop-1<df−/−>) mouse pituitary samples, as well as in the murine pre-somatotrophic GHFT1-5 cell line, see Lew et al., Genes Development 7, 683, consistent with a developmental role for Zn-16.

To demonstrate an aspect of the invention, comparison between the GH-altered Ames dwarf (Prop-1<df−/−>), Snell dwarf (Pit-1<dw^{J}−/−), little (GHRHR, <lit−/−>) transgenic GHRH excess (MT, GHRF, Bri 11) and normal littermate mouse pituitaries showed predicted changes in GH mRNA, as well as other changes in hormone products that had not been previously evaluated or reported. Such quantitative analysis suggests that changes in the regulation of factors functioning in the GH homeostatic regulatory system are consistent with a role for Zn-16 in GH transcriptional control.

Animals, Cell Culture and Isolation of RNA. Normal and Ames dwarf mice maintained in a breeding colony were sacrificed by carbon dioxide euthanasia. The day of birth (by 1400 hrs) was termed day 1. Adult rat pituitaries were obtained by the same procedure. Pituitaries were collected on dry ice, then stored at −70° C. until extracted. GHFT1-5 cells were obtained from Dr. Pamela Mellon (Univ. of California, San Diego), and cultured as described, see Lew et al., supra. Pituitaries and cells were first homogenized (Tissue Tearor, BioSpec, Bartlesville Okla.) in extraction buffer. RNA extraction was performed using the guanidinium-isothiocyanate:phenol:chloroform method as modified by the manufacturer (RNAzol B or Ultraspec, Biotecx, Houston, Tex.). Purity and concentration were checked by UV spectrophotometry (GeneQuant, Pharmacia, Piscataway N.J.).

Reverse Transcription. The RNA pellet was briefly dried before resuspension in 0.1 μM oligo-dT for subsequent reverse transcription of poly-A RNA using the SuperScript II Preamplification System (Life Technologies, Gaithersburg, Md.) according to the manufacturer's instructions. The reaction was carried out in a 20 μl volume containing 200 units of MMLV reverse transcriptase at 45° C. for 60 minutes, then the enzyme was denatured by heating at 65° C. for 15 minutes. Hybridized RNA was removed by digestion with 2 units of E. coli RNase H. The volume was then brought to 50 μl with diethylpyrocarbonate-treated water and stored at −20° C. UV absorbance at 260 nm was measured on a GeneQuant spectrophotometer (Pharmacia, Piscataway, N.J.) in a 50 μl cuvette.

Primer Design. The sequence of rat Zn-15 (accession number L23077) was obtained from GenBank. Prospective primer pairs were computer designed (Right Primer 1.2, BioDisk, San Francisco Calif. or Oligo 4.0, National Biosciences, Plymouth Minn.) using the following criteria: location with respect to zinc fingers, match of Tₘ between the primers, possible secondary structure within the primers, self-hybridization, and hybridization between primers. Primers were synthesized, quantified by UV spectrophotometry (Pharmacia GeneQuant), and stored desiccated at −70° C.

PCR Amplification. Standard PCR conditions for cDNA amplification included each dNTP at a concentration of 0.2 mM and 1.25 U of Taq polymerase in a final volume of 50 μl. Amplifications were performed in a Model TC-1 thermal cycler (Perkin Elmer-Applied Biosystems, Foster City, Calif.). Amplification reactions were initiated with a hot-start using wax beads (Perkin Elmer) to separate the primers, MgCl₂, and dNTPs (Lower master mix) from the DNA template and Taq polymerase (Upper master mix). Thermal cycling conditions consisted of initial denaturation for 60 s at 94° C.; followed by a 3-step profile (94° C. for 60 s; 54° C. for 45 s; and 72° C. for 90 s) for the desired number of cycles, and a terminal extension step of 72° C. for 5 min. For some reactions, a final, non-template dependent extension was carried out at 60° C. for 30 min.

Sequencing Methods, Strategy and Analysis. PCR products from amplification reactions were cloned according to the manufacturer's instructions (TA Cloning Kit, InVitrogen, San Diego Calif.). Ligation reactions were incubated at 14° C. overnight, and then transformed into E. coli INValphaF cells (InVitrogen). Colonies with putative inserts were cultured overnight in 2×YT or TB containing 100 μg/ml ampicillin (Sigma). Bacteria were lysed and plasmid DNA was isolated with a commercial DNA binding matrix (PERFECTprep Plasmid DNA kit, 5 Prime->3 Prime, Boulder Colo.). Restriction digestion with EcoRI followed by agarose gel electrophoresis was used to confirm the presence of inserts prior to sequencing. DNA sequencing reactions were performed using 400 ng of plasmid template and fluorescent dye-labeled dideoxy terminators with AmpliTaq FS DNA polymerase according to the manufacturer's protocol (PRISM Ready Reaction DyeDeoxy Terminator kit, Applied Biosystems, Inc., Foster City Calif.). Thermal cycler conditions were 30 sec. at 96° C., 15 sec. at 50° C., and 4 min. at 60° C. for a total of 25 cycles. Unreacted fluorescent dye-labeled dideoxy terminators were removed from the sequencing reactions using size exclusion gel columns (CentriSep, Princeton Separations, Inc., Adelphia N.J.). Sequencing reactions were electrophoresed on an automated sequencer (Applied Biosystems Model 373A) using a 36 cm well-to-read 6% acrylamide gel (Sooner Scientific, Garvin Okla.) at 28 watts constant power for 10 hours. Data collected on a Macintosh computer were evaluated for ambiguities and “clear” sequence length using Factura 1.2.Or6 software (Applied Biosystems, Inc.). Sequences were aligned using GeneWorks 2.45 software (Oxford BioMolecular, San Diego Calif.) and remaining ambiguities in the electropherograms were resolved from overlapping information, manual inspection, or resequencing. Nucleotide and amino acid sequence data were analyzed, aligned and compared with reported sequences using GeneWorks and Statistical Analysis of Protein Sequences. See Brendel et al., Proc. Natl. Acad. Sci. USA 89, 2002.

Ribonuclease Protection Assay. Total RNA extracted from pituitaries or cells was subjected to ribonuclease protection assay using non-radioactive probe synthesis and detection according to the manufacturer's protocol (RPA II/BrightStar systems, Ambion, Austin Tex.). A 651 nt probe from positions 840 to 1490 of mZn-15 was employed, and a 250 nt probe for mouse beta-actin was used as a control. Chemiluminescence after substrate treatment was visualized on x-ray film (Fuji RX) and then quantified using constant intensity illumination (FotoDyne, Madison Wis.), a CCD video camera (Hamamatsu C2400) or scanner (Nikon) and computer assisted image analysis (Gel-Pro Analyzer, Media Cybernetics).

Mouse Zn-16 cDNA Isolation and Characterization. In order to obtain a probe for mouse (m) Zn-16, mouse pituitary RNA was reverse transcribed using an oligo-dT primer and then amplified using gene-specific oligonucleotide primers which annealed to zinc fingers 1× and XI of rat (r) Zn-15, the region that binds to the GH promoter. Cloned products near the expected size of 506 bp in the rat were found to have a similar DNA sequence, and were used as probes in hybridization screens of a mouse pituitary cDNA library previously constructed in the isolation of mLIM-3. See Seidah et al., DNA 13, 1163. However, no positive colonies were found through several rounds of hybridization. Therefore, the majority of the coding region of mZn-16 cDNA was isolated by amplification using primers specific for different zinc fingers of rZn-15, and the 5′ and 3′ ends were isolated using rapid amplification of cDNA ends.

Six portions of mZn-16 of at least 1500 bp were independently amplified, cloned and sequenced. Sequences were obtained in both directions from multiple clones of each fragment, and then aligned to assemble the entire cDNA sequence of 6879 nucleotides. The full-length mZn-16 cDNA sequence was assembled from overlapping regions of at least 1500 bp.

The open reading frame in the mZn-16 cDNA as shown in SEQ ID:NO 1 encodes a polypeptide as shown in SEQ ID:NO 2. The mZn-16 amino acid sequence of 2292 amino acids has several additional aa that are not present in rZn-15, particularly in the N-terminus. There are four in-frame methionine residues upstream from the initial methionine in the rat, for additional translational start sites not reported in the rat. A feature of the protein that has been identified based on computer analysis are several regions (aa 830-845, 1550-1567, 1999-2016) encoding consensus eukaryotic nuclear localization signals, see Robbins et al., Cell 64, 615.

Importantly, multiple zinc fingers of the Cys2His2-type, see Berg and Shi, Science 271, 1081, similar to those found in rZn-15, are present in mZn-16. There is 97% amino acid identity between the zinc fingers of rZn-15 and mZn-16. However, the four differences in mZn-16 in finger V change three proline residues. This region of mZn-16 is now predicted to contain two consensus zinc fingers, designated as Va and Vb in FIG. 2B, and finger Va now agrees at all positions with the zinc finger consensus sequence. Changed residues in fingers II, IX, X and XIII are conservative substitutions, and substitutions in regions outside the fingers would not be predicted to have any impact on zinc coordination or DNA binding, see Berg and Shi, Science 271, 1081. As found in rZn-15, there are extended linker regions between the zinc fingers in mZn-16, particularly in the C-terminal half of the protein. The largest region of consecutive difference between mouse Zn-16 and rat Zn-15 is found in the linker between fingers VII and VIII (aa 845 to 860 in mZn-16), where only two of the 14 consecutive residues are similar.

Mouse Zn-16 mRNA expression in Normal and Ames dwarf pituitaries. Zn-16 expression in normal and GH-deficient Ames dwarf (Prop-1<df−/−>) mouse pituitaries was studied at the day of birth (postnatal day one). Total RNA was isolated, reverse transcribed using oligo-dT priming, then amplified using primers in fingers IX-XI for 30 cycles. The amount of 503 bp product of these amplifications was then determined using image analysis of ethidium bromide-stained gels. Samples in which no pituitary cDNA was added as control showed no amplification. The average relative image intensity of the bands was determined for normal and Ames dwarf mice (n=3). At one day of age, there was no significant difference in the expression of Zn-16 between normal and Ames dwarf pituitary.

Mouse Zn-16 mRNA Expression in GHFT1-5 pre-somatotroph cells. The expression of Zn-16 mRNA in Ames dwarf pituitaries suggested that Zn-16 might be expressed in the mouse cell line GHFT1-5, which is derived by immortalization of pituitary cells with a Pit-1 promoter-driven large T antigen construct. These cells have been characterized as pre-somatotrophs, but they do not express Pit-1 protein or GH mRNA. Probes were produced to measure the expression of mZn-15 mRNA in total RNA using ribonuclease protection assays (RPA). Total RNA was isolated either from ca. 30 pooled mouse pituitaries or 107 GHFT1-5 cells, and used for RPA for either Zn-16 or actin mRNA in each sample. Actin mRNA was assayed to serve as a control for each sample in a 5 μg RNA aliquot. The size of the protected fragment for Zn-16 was the same in both pituitary and GHFT1-5 samples, and was of the size predicted from the length of homologous probe sequences (651 nt). In comparing the pituitary and GHFT1-5 samples, the detectable amounts of mZn-16 were different, suggesting that Zn-16 expression levels varied between normal pituitary and GHFT1-5 samples.

The further description found immediately below shows Zn-16 function in the transcriptional regulatory control of pituitary GH expression.

Animal care and use. Male mice 3-4 months old were used in this study. Mice were euthanized with carbon dioxide anesthesia, then individual pituitaries were removed using washed instruments and stored in single tubes at −70° C.

RNA preparation. Total RNA was extracted from individual mouse pituitaries using a modified phenol/chloroform/guanidinium protocol (Ultraspec; Biotecx, Houston, Tex.). The RNA pellet was briefly dried before resuspension in 0.1 μM oligo-dT for subsequent reverse transcription of poly-A RNA using the SuperScript II Preamplification System (Life Technologies, Gaithersburg, Md.) according to the manufacturer's instructions. The volume was brought to 50 μl with diethylpyrocarbonate-treated water for storage at −20° C. UV absorbance at 260 nm was measured on a GeneQuant spectrophotometer (Pharmacia, Piscataway, N.J.) in a 50 μl cuvette.

PCR. Primers were selected for specificity for mouse mRNAs, high annealing temperature, and absence of secondary structure using the computer programs Right Primer (BioDisk, San Francisco, Calif.) and Oligo 5.0 (National Biosciences, Plymouth, Minn.). Standard cDNA amplification reactions included each dNTP at a concentration of 0.2 mM and 1.25 U of Taq polymerase in a final volume of 50 μl. The fluorescent dye-labeled dUTPs ([F]dUTP) used for labeling were [R110], [R6G], or [TAMRA] (ABI; Foster City, Calif.). [F]dUTPs were diluted for a constant addition volume of 0.1 μl per reaction. Amplifications were performed in a Model TC-1 thermal cycler (ABI). Amplification reactions were initiated with a hot-start using AmpliWax PCR Gem 50 beads (ABI). Thermal cycling conditions consisted of initial denaturation for 60 s at 94° C.; followed by a 3-step profile (94° C. for 60 s; 54° C. for 45 s; and 72° C. for 90 s) for the desired number of cycles, and a terminal extension step of 72° C. for 5 min. For some reactions, a final, non-template dependent extension was carried out at 60° C. for 30 min. After amplification, unincorporated primers and dNTPs were removed by centrifugation through Centricon-50 or Microcon-30 filters (Amicon, Beverly, Mass.) filters. Products were analyzed on 12 cm well-to-read (WTR) gels composed of 10% Long Ranger (FMC BioProducts, Rockland, Me.) with a 373A instrument using GeneScan 1.2 software. Electrophoresis was performed with power limiting at 12W in 0.5×TBE buffer. Samples were mixed with a ROX-dye labeled size marker (ROX-500, ABI) and a sucrose- or Ficoll-bromophenol blue dye solution before loading on the gel. Results were statistically analyzed using the computer programs Excel (Microsoft, Redmond, Wash.) and SuperANOVA (Abacus Concepts, Berkeley, Calif.).

Comparison of transcript abundance. In order to compare product intensities among a large number of amplification reactions, the fluorescence detection and sizing capacity of an automated sequencing instrument was used. Amplifications were performed in the presence of fluorescence-labeled dUTP, which was detectable during electrophoresis. Fluorescence labeled products were examined for molecular weight compared to the standards present in each lane as an internal control, and for peak intensity from the processed electropherograms. From peak heights and the calculated efficiency, the transcript abundance was determined as related by the equation in Gilliland et al., Proc. Natl. Acad. Sci. USA 87, 2725. For normal littermates and GH-affected mice, pituitaries were amplified for GH, Zn-16 and Pit-1 abundance. The results, expressed as percentage of normal littermate expression, were:

| mouse type | GH | Zn-16 | Pit-1 |
|---|---|---|---|
| Ames dwarf | 0 | 4.5 | 0 |
| Snell dwarf | 0 | 5.9 | 0.2 |
| Little | 1.3 | 5.9 | 2.9 |
| GHRH giant | 242.4 | 198.2 | 199.8 |

Simple regression correlation tests showed that there was a significant correlation (p<0.001) for Pit-1 vs. GH (F_{1.17}, 26.82) and Zn-16 vs. GH (F_{1.17}=12.48). This correlation from an in vivo model implicates Zn-16 function in the transcriptional regulatory control of pituitary GH expression.

Further embodiments of aspects of the invention are described below.

In an embodiment of an aspect of the invention, a Zn-16-expressing construct with inducible control by exogenous factors (e.g., tetracycline) is stably transfected into mammalian cells (e.g., GC cells). The transfected cells are placed within a permeable membrane for immunological protection (“hollow fiber”). The hollow fiber containing the transfected cells is implanted into the kidney capsule of a patient who has undergone surgery. The cells within the hollow fiber express GH regulated by the Zn-16 which is induced by the physician (e.g., with tetracycline) as needed to promote healing. In a preferred embodiment, localized administration of GH expression is provided by implantation of the hollow fiber unit during the surgery, where compatible with needed removal processes. Such an embodiment of the invention is useful for enhancing GH production, desired in particular during the healing process, promoting tissue regeneration and lessening the need for invasive repetitive injections.

In another embodiment of an aspect of the invention, a Zn-16-expressing construct with inducible control by exogenous factors (e.g., tetracycline) is stably transfected into mammalian IQ cells (e.g., GC cells). The transfected cells are placed within a permeable membrane for immunological protection (“hollow fiber”). The hollow fiber containing the transfected cells is implanted into the kidney capsule of a patient who has experienced severe burns. The cells within the hollow fiber express GH regulated by the Zn-16 which is de-induced by the physician (e.g., with tetracycline) after promoting healing. Such an embodiment of the invention is useful for enhancing GH production, desired in particular during the healing process, promoting tissue regeneration and lessening the need for invasive repetitive injections.

In yet another embodiment of an aspect of the invention, a Zn-16-expressing construct with inducible control by exogenous factors (e.g., tetracycline) is stably transfected into mammalian cells (e.g., GC cells). The transfected cells are placed within a permeable membrane for immunological protection (“hollow fiber”). The hollow fiber containing the transfected cells is implanted into the kidney capsule of a patient who has experienced muscle wasting, as is observed, for example, in later-stage HIV-infected patients. The cells within the hollow fiber express GH regulated by the Zn-16 which is subject to control by the physician (e.g., with tetracycline) for the optimal control of healing and the lessening of the pace of muscle wasting. Such an embodiment of the invention is useful for enhancing GH production, desired in particular during the healing process, promoting tissue regeneration and lessening the need for invasive repetitive injections.

In certain embodiments of the invention, Zn-16 is administered to a patient. As used in this application, “administration” includes the implantation of a construct or a host cell containing a Zn-16 nucleic acid sequence or, more generally, encoding the Zn-16 polypeptide, for instance as described above. That is, administration of Zn-16 is effected also by the administration to a patient, or the implantation into a patient, of a construct encoding the Zn-16 polypeptide.

In an embodiment of an aspect of the invention, Zn-16 is used to alter gene expression in a patient who has a pituitary tumor. In a preferred embodiment, Zn-16 and somatostatin are co-administered to the patient. Upon administration, the somatostatin binds its receptor, is internalized, and the Zn-16 alters gene expression in such a way as to ameliorate the effects of the tumor.

In another embodiment of an aspect of the invention, Zn-16 is used to control expression of proteins whose expression is otherwise driven by other zinc finger proteins. In a particular embodiment, Zn-16 is administered to a patient suffering from a cancer that is characterized by the overexpression of proteins whose expression is driven by zinc finger proteins other than Zn-16. The administered Zn-16 blocks the overexpression driven by other zinc finger proteins and slows tumor growth.

In another embodiment, Zn-16 is affixed to a surface. The presence of heavy metals is detected by their binding to the affixed Zn-16. Methods of the detection of subtle changes in binding, such as surface plasmon resonance or methods capable of detection of small changes in potential difference across a very limited space, enable the detection of very low concentrations of heavy metals when the metals bind Zn-16. In a particular embodiment, Zn-16 is preloaded with one heavy metal, and displacement of that heavy metal is measured.

In another embodiment, Zn-16 is used as a heavy-metal responsive factor that indicates contamination levels. In such an embodiment, Zn-16-driven expression of a reporter gene changes when there is a change in the heavy metal concentration. Changes in the concentration of not only zinc but also other heavy metals such as mercury and lead are detected.

In yet another embodiment, the heavy-metal-binding property of Zn-16 is exploited to ameliorate the adverse effect on a patient of heavy metal exposure. By virtue of its heavy-metal-binding property, Zn-16 serves as a sink that binds excess heavy metal that is toxic to key tissues in the patient, such as liver and kidney.

In another embodiment, Zn-16 is pre-administered to a human to prevent the adverse effect on the human of anticipated heavy metal exposure. A human anticipating participation in cleanup of heavy metal environmental contamination, for example from a spill of stored liquids or from radioactive fallout, self-pre-administers Zn-16, which binds environmental heavy metals to lessen harm to the human. The Zn-16 is a reservoir for the metals which otherwise pose a danger to the human.

In another embodiment of an aspect of the invention, Zn-16 is administered to a human patient who has diabetes or, more generally, experiences dysregulation of expression or availability of insulin or insulin-like growth factor. The administered Zn-16 regulates the expression of GH to alter levels of insulin and insulin-like growth factor and ameliorates the patient's condition.

In an embodiment of an aspect of the invention, Zn-16 is used in a method of controlling the expression of gene products at a multiplicity of loci in the genome of an organism, said method comprising: the site-directed or random mutagenesis of Zn-16 to encode an altered polypeptide, said altered polypeptide possessing an affinity for a transcriptional regulatory factor different from the affinity of the polypeptide of Zn-16 for said transcriptional regulatory factor; the formation of an assemblage of said transcriptional regulatory factor with said altered polypeptide; and the direction of said expression at said loci by said assemblage. In a particular embodiment, the altered polypeptide binds a multiplicity of transcriptional regulatory factors to form the assemblage. In a particularly preferred embodiment, the method controls the expression of gene products throughout the entire genome of the organism, allows specific targeting of gene promoters, and creates a controllable platform to assemble other transcriptional regulatory factors.

In another embodiment, Zn-16 is modified to have temperature-responsive zinc binding. This modified Zn-16 is useful as a temperature-dependent regulator of gene expression.

In another embodiment, a cell or tissue sample is taken from a patient. Nucleic acids from the patient are extracted from the sample. Hybridization of the patient's nucleic acids to the nucleic acid encoding Zn-16 is measured. Detection of a difference between the hybridization of the patient's sample and the hybridization of a control sample is useful in the diagnosis of dwarfism, gigantism, hypothyroidism, and other disorders of metabolism.

The invention is not limited to the exact details of operation, or to the exact compositions, methods, procedures, or embodiments shown and described, as obvious modifications and equivalents will be apparent to one skilled in the art. As the invention would therefore be limited only by the full scope which could reasonably, legally and equitably be accorded any of the appended claims, the foregoing examples are provided merely by way of illustration of the breadth of the present invention, which exceeds any and all of these examples.

I claim:

1. An isolated nucleic acid comprising the nucleotide sequence of SEQ ID NO: 1, or of a degenerate variant of SEQ ID NO:1.

2. An isolated nucleic acid comprising a sequence that encodes a polypeptide having the sequence of SEQ ID NO:2, or of SEQ ID NO:2 with conservative amino acid substitutions.

3. A nucleic acid construct comprising the nucleic acid of claim 1 operably linked to a heterologous flanking sequence.

4. A nucleic acid construct comprising the nucleic acid of claim 1 operably linked to a heterologous transcriptional regulatory sequence.

5. A nucleic acid construct comprising the nucleic acid of claim 1 operably linked to a heterologous coding sequence.

6. A host cell comprising the heterologous nucleic acid of claim 1.

7. A method of detection of a nucleic acid comprising measurement of the extent of hybridization of said nucleic acid to the nucleic acid of claim 1.

8. A method of controlling the expression of a gene comprising the step of binding a transcriptional regulatory molecule to the nucleic acid construct of claim 3.

9. An isolated polypeptide comprising the amino acid sequence of SEQ ID NO:2, or of SEQ ID NO:2 with conservative amino acid substitutions.

10. A method of controlling the expression of gene products at a multiplicity of loci in the genome of an organism, said method comprising: the site-directed or random mutagenesis of the polypeptide of claim 9 to form an altered polypeptide, said altered polypeptide possessing an affinity for a transcriptional regulatory factor different from the affinity of the polypeptide of claim 9 for said transcriptional regulatory factor; the formation of an assemblage of said transcriptional regulatory factor with said altered polypeptide; and the direction of said expression at said loci by said assemblage.

11. A method of identifying an agent that changes the level of expression of a growth hormone gene, said method comprising: combining said agent with the nucleic acid construct of claim 5; and observing the resultant change in transcription of said growth hormone gene.

12. A method of controlling the rate of growth of an organism, said method comprising the introduction of the nucleic acid construct of claim 3 into said organism, said introduction altering the endogenous regulation of the expression of an endogenous growth hormone gene in said organism.

13. A method of controlling the rate of growth of an animal, said method comprising the introduction of the nucleic acid construct of claim 3 into said animal, said introduction altering the endogenous regulation of the expression of an endogenous growth hormone gene in said animal.

14. A method of controlling the rate of growth of a vertebrate animal, said method comprising the introduction of the nucleic acid construct of claim 3 into said vertebrate animal, said introduction altering the endogenous regulation of the expression of an endogenous growth hormone gene in said vertebrate animal.

15. A method of controlling the rate of growth of a mammal, said method comprising the introduction of the nucleic acid construct of claim 3 into said mammal, said introduction altering the endogenous regulation of the expression of an endogenous growth hormone gene in said mammal.

16. A method of controlling the rate of growth of a mouse, said method comprising the introduction of the nucleic acid construct of claim 3 into said mouse, said introduction altering the endogenous regulation of the expression of an endogenous growth hormone gene in said mouse.

17. The method of claim 10 wherein the altered polypeptide binds a multiplicity of transcriptional regulatory factors to form the assemblage.

18. A method of controlling the expression of gene products throughout the entire genome of an organism, said method comprising: the introduction of an alteration to the polypeptide of claim 9 to form an altered polypeptide; the assembly of a multiplicity of transcriptional regulatory factors on the altered polypeptide to form a controllable platform; and the specific targeting of the controllable platform to a plurality of promoters in said genome.

19. The method of claim 7 wherein the nucleic acid being detected is taken from a human subject and wherein the hybridization measured is compared with the hybridization of another nucleic acid taken from a human subject to the nucleic acid of claim 1.

20. A method of treating a human patient in whom it is desired to regulate metabolic rate or growth comprising the administration to said patient of a therapeutically effective amount of the nucleic acid of claim 1.
